(19) United States
(12) Patent Application Publication
(10) Pub. No.: US 2005/0137610 A1
(43) Pub. Date: Jun. 23, 2005

(54) Facilitating catheter assembly

(76) Inventor: Arnold Miller, Chestnut Hill, MA (US)

Correspondence Address: Mark J. Pandiscio;Padiscio & Pandiscio, P.C., 470 Totten Pond Road, Waltham, MA, 02451-1914, US

(21) Appl. No.: 10/818,012

(22) Filed: Apr. 5, 2004

Related U.S. Application Data
(60) Provisional application No. 60/460,198, filed on Apr. 4, 2003.

(51) Int. Cl. A61F 11/00

(52) U.S. Cl. 606/108; 604/163

(57) ABSTRACT

A method for advancing a working instrument through the vascular system of a patient, the method comprising the steps of: advancing a facilitating catheter assembly within the vascular system of a patient, wherein the facilitating catheter assembly is formed out of a implantable graft material; and advancing the working instrument through the facilitating catheter assembly. A facilitating catheter assembly for use in for advancing a working instrument through the vascular system of a patient, the facilitating catheter assembly comprising a facilitating sheath and a companion sheath, the facilitating sheath being disposed within the companion sheath, the companion sheath including means for opening the companion sheath so as to release the facilitating sheath, and the facilitating sheath being formed out of implantable graft material. A method for advancing a working instrument through the vascular system of a patient, the method comprising the steps of: advancing a facilitating catheter assembly within the vascular system of a patient, wherein the facilitating catheter assembly is constructed so as to (i) have a first diameter which is less than the diameter of the working instrument, and (ii) be capable of expanding to a second diameter which is at least as large as the diameter of the working insturment; and advancing the working instrument through the facilitating catheter assembly. A facilitating catheter assembly for use in advancing a working instrument through the vascular system of a patient, the facilitating catheter assembly being constructed so as to (i) have a first diameter which is less than the diameter of the working instrument, and (ii) be capable of expanding to a second diameter which is at least as large as the diameter of the working instrument.

### REFERENCE TO PENDING PRIOR PATENT APPLICATION

This patent application claims benefit to pending prior U.S. Provisional Patent Application Ser. No. 60/460,198, filed Apr. 4, 2003 by Arnold Miller for FACILITATING CATHETER ASSEMBLY (Attorney's Docket No. AM-6 PROV), which patent application is hereby incorporated herein by reference.

### FIELD OF THE INVENTION

This invention relates to surgical procedures in general, and more particularly to endoluminal procedures for repairing aortic aneurysms and other procedures requiring endoluminal access to the aorta.

### BACKGROUND OF THE INVENTION

Currently, when an endoluminal approach is to be used to repair an aortic aneurysm 5 (FIG. 1), or to otherwise gain access to the aorta via the femoral artery, a small guidewire (not shown) is first advanced up the femoral artery 10, up the iliac branch 15 and up into the aorta 20. Then a delivery catheter (not shown), containing the stent (not shown) which is to be used to repair the aneurysm, is advanced up the guidewire to the site of the aneurysm, where the stent is deployed.

Unfortunately, the iliac branch 15 can frequently be very tortuous, stenotic and/or occluded, or the diameter may be small such that it can be quite difficult or even impossible to pass the relatively large delivery catheter up the iliac branch, even with the help of the guidewire. Furthermore, as the surgeon forces the relatively large delivery catheter up the iliac branch, the surgeon runs the risk of seriously damaging or even rupturing the blood vessel. Thus, in some circumstances the surgeon may be forced to abandon the effort to advance the delivery catheter up the iliac branch to the aorta.

In connection with the foregoing, it should also be appreciated that the delivery catheter is a relatively expensive device, commonly on the order of $10,000 or so. As a result, it can be an expensive “dead end” where the surgeon opens up the delivery catheter package, tries to insert the delivery catheter into patient and then fails—if this happens, the surgeon has effectively wasted $10,000. Thus, the surgeon would prefer to open the delivery catheter package only where the surgeon has a high degree of confidence that it will be possible to advance the delivery catheter through the iliac branch to the site of the aneurysm.

In view of the foregoing, where the surgeon has concerns about the ability to pass the delivery catheter through the iliac branch to the surgical site, it is common for the surgeon to first pass the small guidewire up to the site of the aneurysm, and then pass a relatively large, relatively rigid test sheath up the guidewire to surgical site. This test sheath has a diameter similar to that of the delivery catheter or slightly larger, so that if the surgeon can successfully advance the test sheath to the surgical site, the surgeon can be fairly certain that they will be able to successfully pass the delivery catheter to the surgical site or to pass the delivery catheter through this test sheath. Of course, if the surgeon is not successful in advancing the test sheath to the surgical site, then the surgeon does not open the package for the delivery catheter.

While the foregoing procedure is fairly reliable in predicting whether the delivery catheter can be successfully advanced to the aneurysm site, it still has a major disadvantage. Specifically, in some cases where the large test sheath is inserted into the iliac branch, there is still the possibility that the blood vessel may be ruptured as the test sheath is advanced. If such a rupture does occur, bleeding from the ruptured vessel may be contained while the test sheath is in place; however, as soon as the test sheath is withdrawn, the patient bleeds, often violently, causing significant trauma to the patient and generally requiring immediate corrective surgery.

In addition to the foregoing, two additional methods may also be used to help pass the delivery sheath into the aorta when the iliac branch is very diseased, calcified or narrowed:
(i) the area of stenosis/narrowing can be ballooned open (i.e., angioplasty) so as to facilitate passage of the relatively large delivery catheter through the iliac branch; and/or
(ii) a series of graduated, progressively-sized dilators can be passed through the iliac branch until a sufficiently large diameter is opened to pass the delivery sheath through the iliac branch.

While both of these methods can help open an otherwise-constricted iliac branch and thus facilitate passing the relatively large delivery catheter to the site of the aneurysm, they also suffer from the fact that they tend to result in an irregular surface on the inside wall of the iliac branch. This is believed to be due to the stretching of the artery and the fracturing of plaque on the inside wall of the artery. Subsequent passage of a delivery catheter (or larger dilator) through such an artery can result in elevation of the lining of the artery (i.e., the creation of a flap) which can cause severe damage to, or even perforate, the artery. In addition, the irregular surface increases friction between the arterial wall and the delivery catheter (or larger dilator), which can make it difficult or even impossible to pass the device through the artery without excessive force being applied. Such excessive force can result in rupture of the artery, with the resulting serious consequences as described above.

In addition to the foregoing, the same types of problems may be encountered while performing similar or other procedures through or on other vascular structures, e.g., the coronary, renal, visceral, cerebral and/or other vessels.

### SUMMARY OF THE INVENTION

These and other objects and features of the present invention are addressed by the provision and use of the invention which, in one preferred form, comprises a method for advancing a working instrument through the vascular system of a patient, the method comprising the steps of:
advancing a facilitating catheter assembly within the vascular system of a patient, wherein the facilitating catheter assembly is formed out of a implantable graft material; and
advancing the working instrument through the facilitating catheter assembly.

In another form of the invention, there is provided a facilitating catheter assembly for use in for advancing a working instrument through the vascular system of a patient, the facilitating catheter assembly comprising a facilitating sheath and a companion sheath, the facilitating sheath being disposed within the companion sheath, the companion sheath including means for opening the companion sheath so as to release the facilitating sheath, and the facilitating sheath being formed out of implantable graft material.

In another form of the invention, there is provided a method for advancing a working instrument through the vascular system of a patient, the method comprising the steps of:
advancing a facilitating catheter assembly within the vascular system of a patient, wherein the facilitating catheter assembly is constructed so as to (i) have a first diameter which is less than the diameter of the working instrument, and (ii) be capable of expanding to a second diameter which is at least as large as the diameter of the working insturment; and
advancing the working instrument through the facilitating catheter assembly.

In another form of the invention, there is provided a facilitating catheter assembly for use in advancing a working instrument through the vascular system of a patient, the facilitating catheter assembly being constructed so as to (i) have a first diameter which is less than the diameter of the working instrument, and (ii) be capable of expanding to a second diameter which is at least as large as the diameter of the working instrument.

### BRIEF DESCRIPTION OF THE DRAWINGS

These and other objects and features of the present invention will be more fully disclosed or rendered obvious by the following detailed description of the preferred embodiments of the invention, wherein like numbers refer to like parts and further wherein:

FIG. 1 is a schematic view of selected anatomy of a patient;

FIG. 2 is a schematic view of a novel facilitating catheter assembly formed in accordance with the present invention, and shown in conjunction with a standard introducer and a guidewire;

FIG. 3 is a schematic view showing the facilitating catheter assembly having its companion sheath opened to release its facilitating sheath;

FIGS. 4-8 show the facilitating catheter assembly of the present invention being used to deliver a delivery catheter;

FIG. 9 is a schematic view showing an endoluminal cutting device which may be used in conjunction with the present invention; and

FIGS. 10A and 10B are schematic views showing a permanent liner/graft deployed within the aorta of a patient.

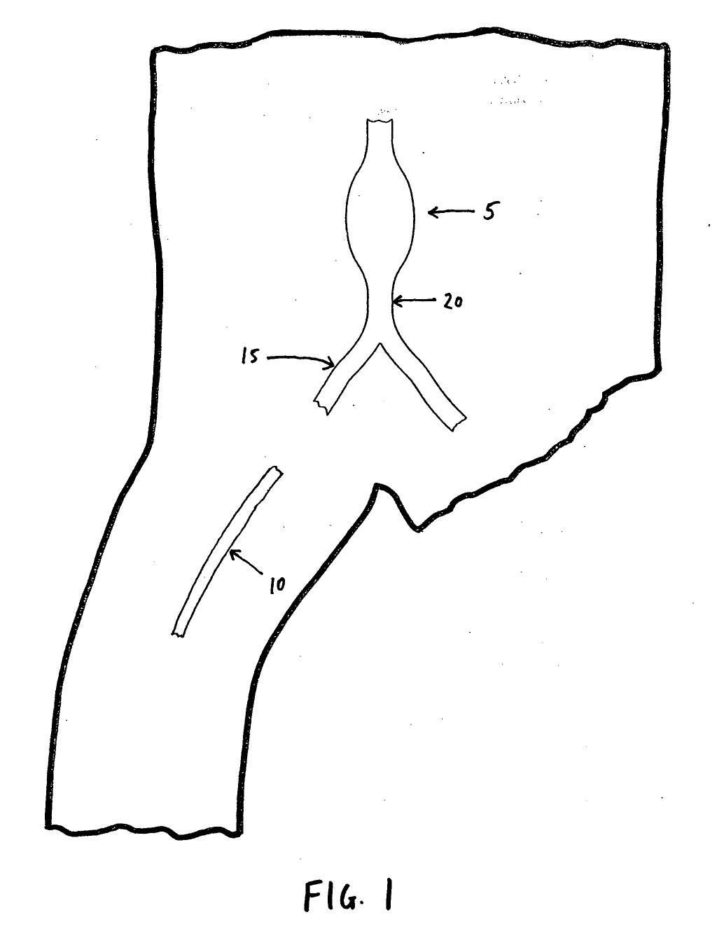

### DETAILED DESCRIPTION OF THE PREFERRED EMBODIMENTS

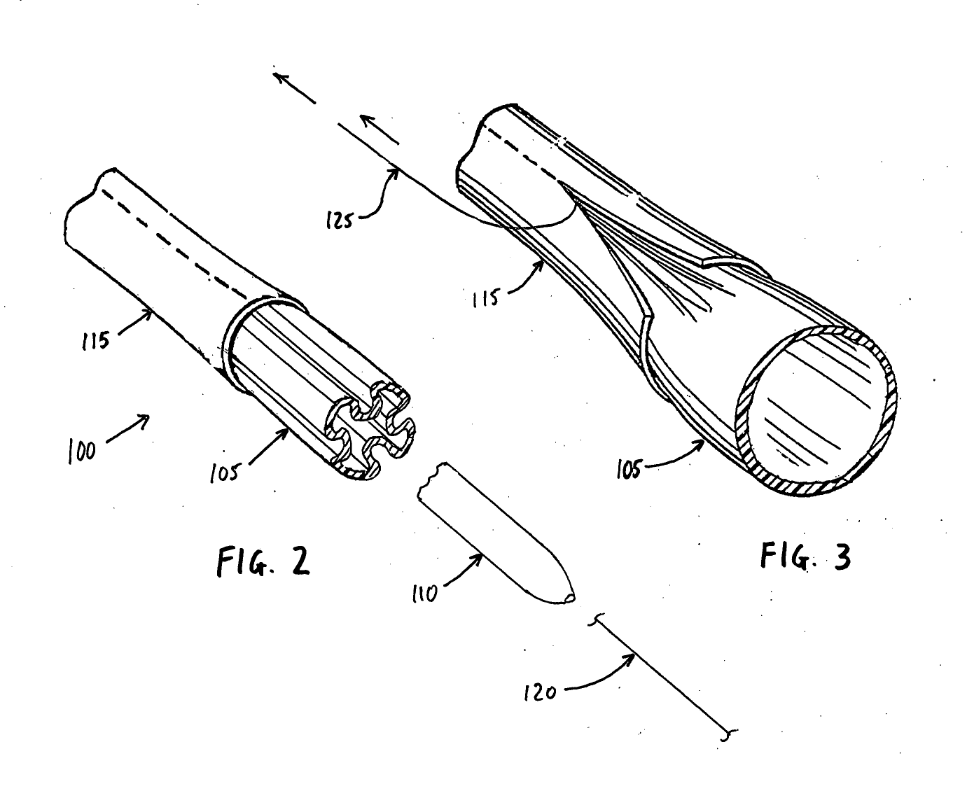

In view of the foregoing, a novel solution has now been developed. More particularly, there has been created a novel facilitating catheter assembly 100 (FIG. 2), which comprises a facilitating sheath 105 which is initially wrapped (e.g., twisted, folded, rolled, etc.) around a standard introducer 110 and held packed inside a companion sheath 115. The facilitating sheath 105 is formed out of a flexible, strong, but very thin-walled tube with a specially coated surface to minimize friction. By way of example but not limitation, facilitating sheath 105 may be made out of implantable “graft” material of the sort used for bypass surgery. The companion sheath 115 is sized so as to have a diameter significantly less than the diameter of the aforementioned delivery catheter, and the facilitating sheath 105 has a diameter (when unfolded) somewhat larger than the delivery catheter, and preferably substantially the same as the blood vessel it is to be received in (see below).

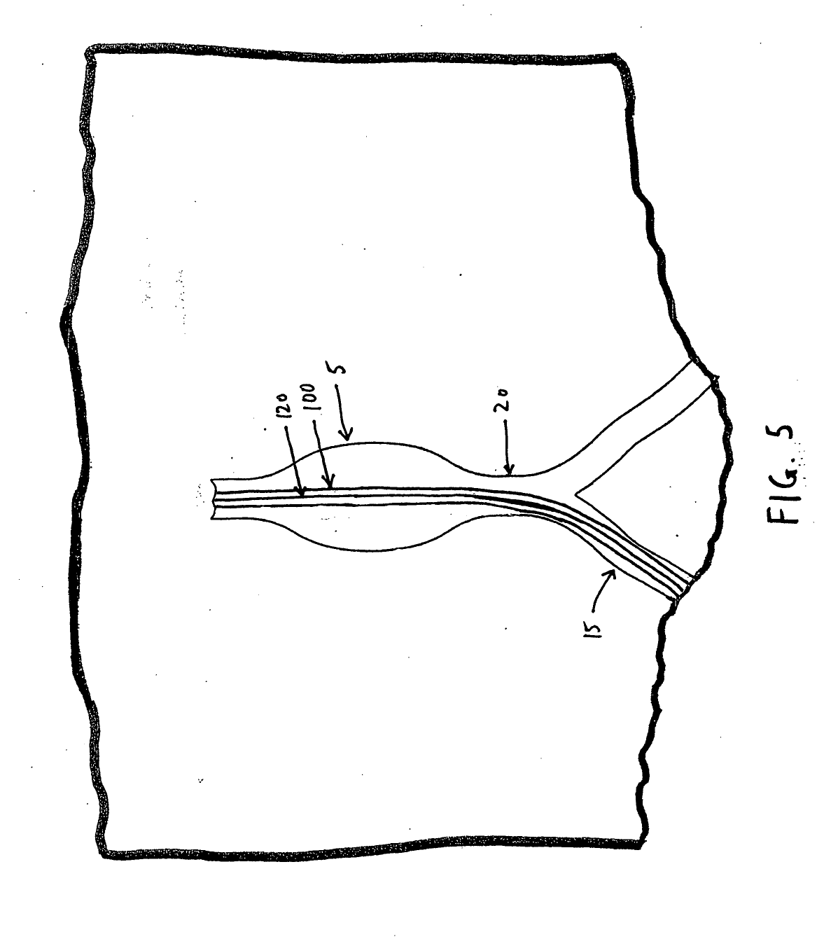

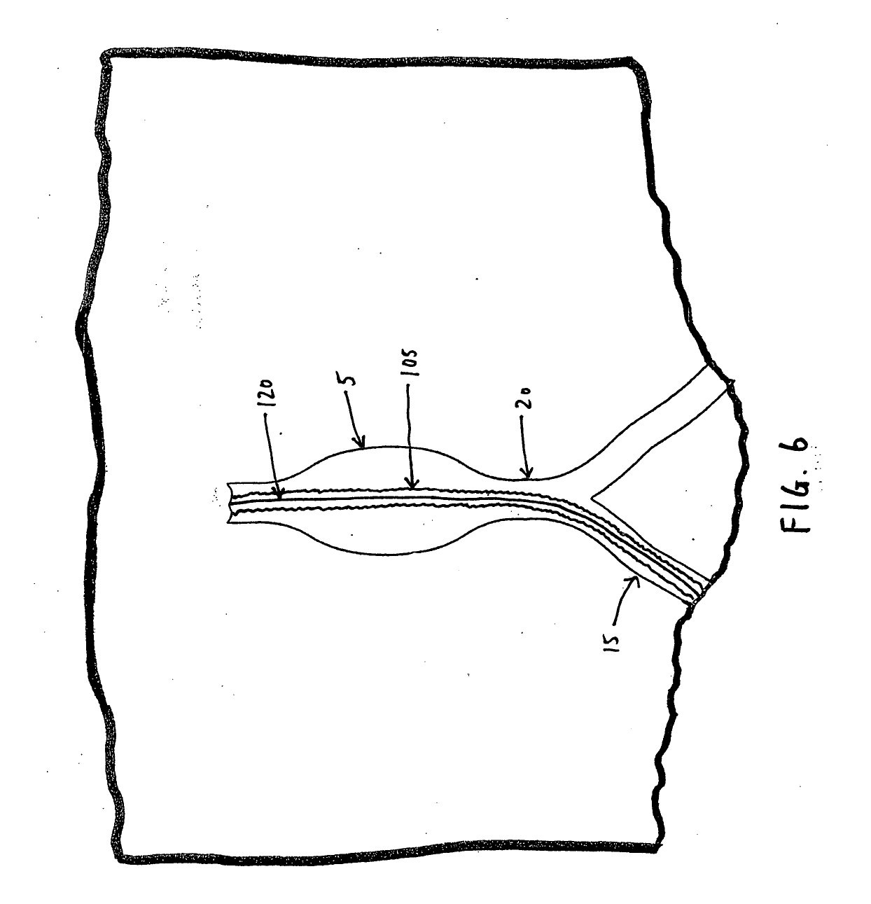

In practice, the guidewire 120 (FIG. 2) is first advanced to the surgical site in the usual manner (FIG. 4). Then the facilitating catheter assembly 100 (i.e., consisting of the facilitating sheath 105 wrapped around a standard introducer 110 and held inside the companion sheath 115) is advanced up the guidewire 120 to the site of the aneurysm (FIG. 5). In this respect it will be appreciated that the facilitating catheter assembly 100 can typically be advanced through the iliac branch without undue difficulty due to the relatively small outer diameter of companion sheath 115.

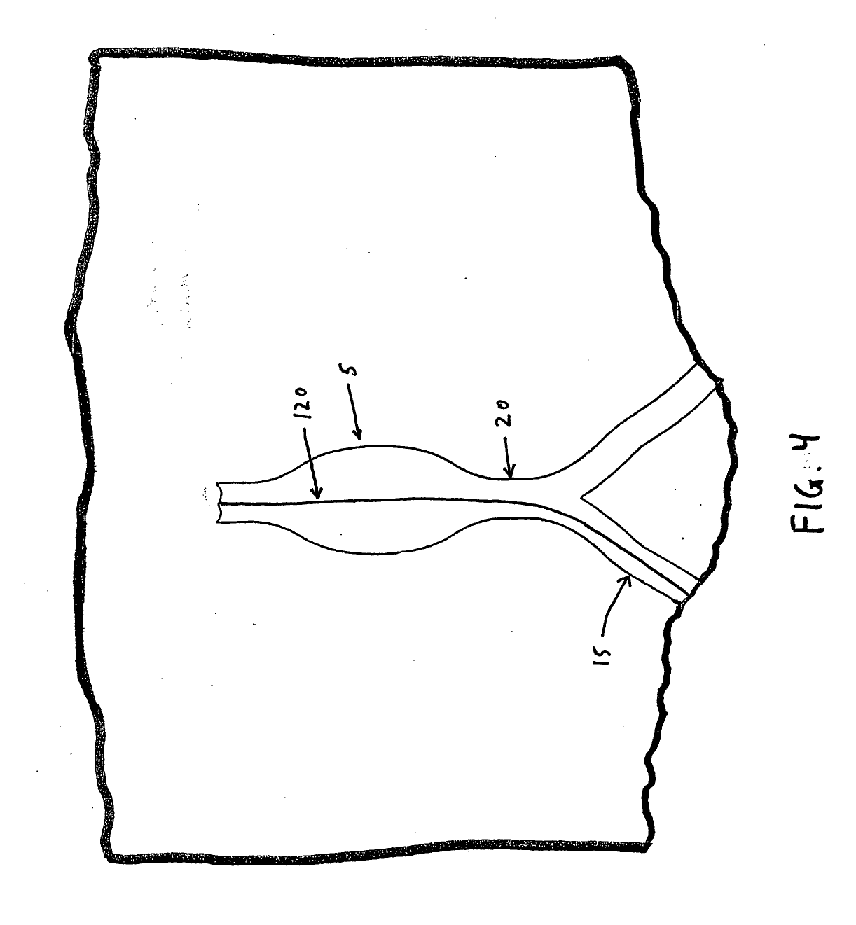

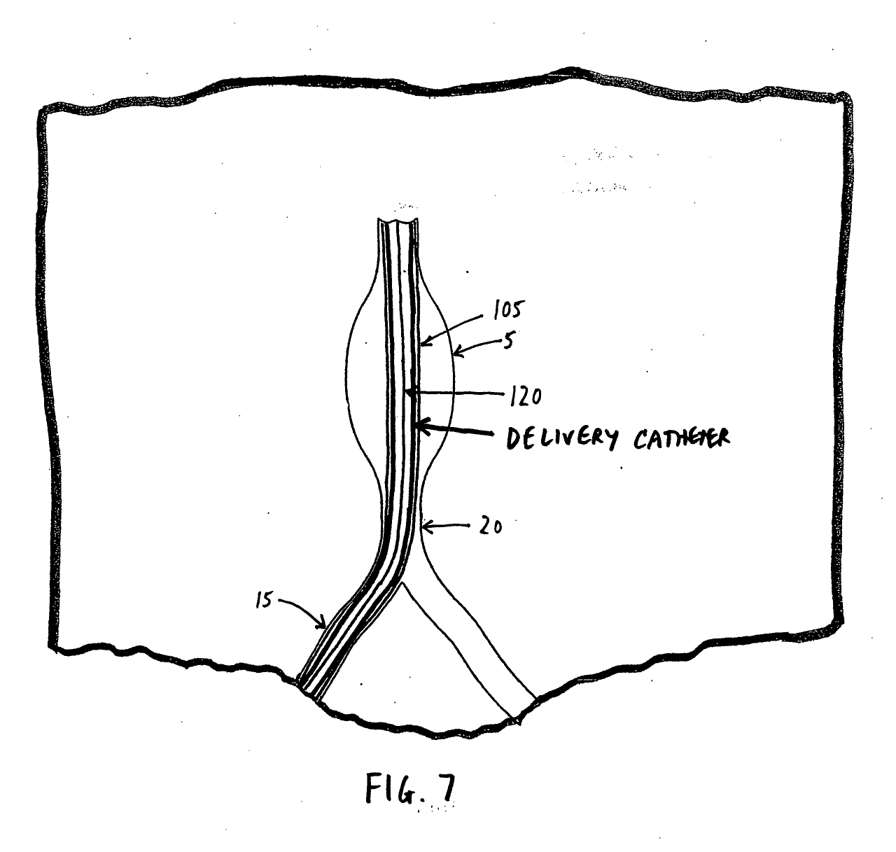

Next, the introducer 110 is removed, followed by removal of the companion sheath 115, e.g., by pulling a tear string 125 (FIG. 3) embedded in the wall of the companion sheath 115, so that it rips along its length and can thereafter be pulled away or, alternatively, a “peel away” companion sheath may be used. This will leave just the guidewire 120 and the facilitating sheath 105 in the blood vessel (FIG. 6).

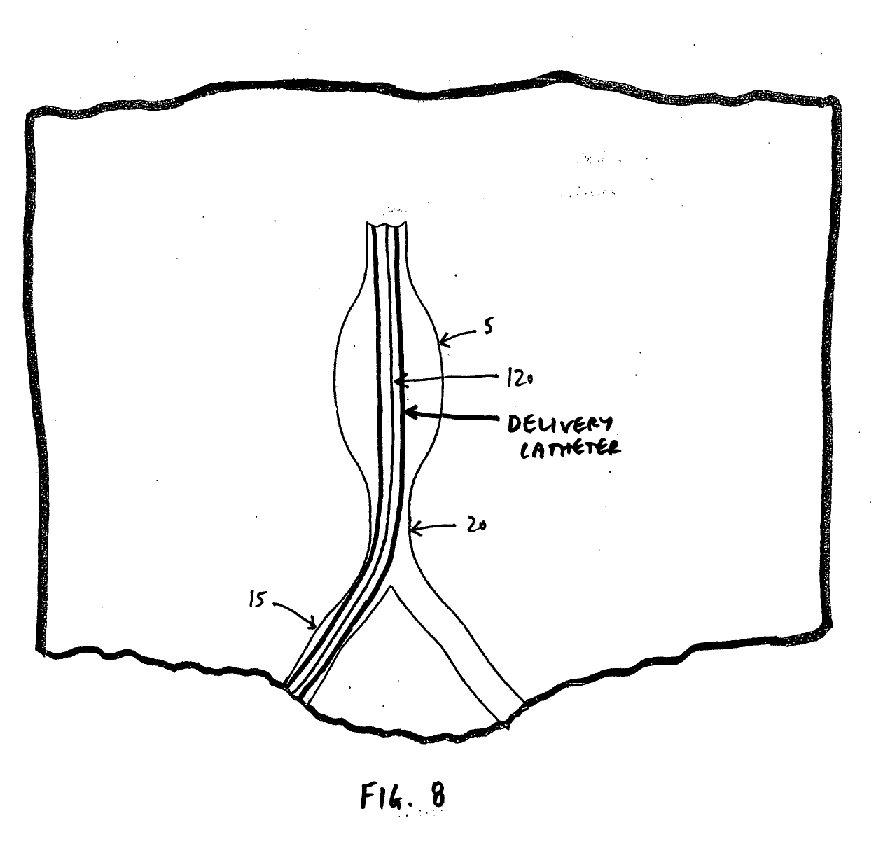

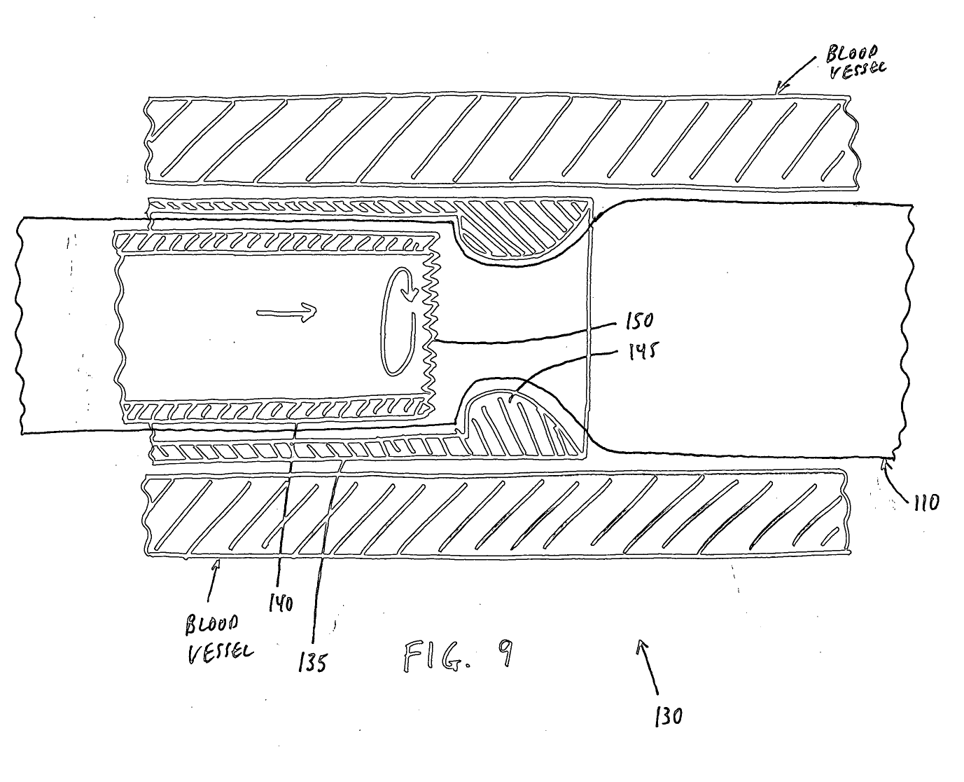

Depending on the circumstances, the aforementioned test sheath and/or the aforementioned balloon angioplasty device and/or the aforementioned progressive dilators and, ultimately, the delivery catheter (containing the stent), may be loaded onto the guidewire 120, introduced into the facilitating sheath 105 and then advanced to the surgical site. As the facilitating sheath 105 receives the one or more devices, the facilitating sheath 105 expands to its full size and acts as a frictionless liner for the advancing device, thereby facilitating passage through the blood vessel (FIG. 7). To the extent that the delivery catheter (carrying the stent) is successfully advanced to the aneurysm site, the facilitating sheath 105 may then be removed and the delivery catheter used to deploy the aneurysm stent (FIG. 8).

If, during the procedure, it is discovered that the facilitating catheter assembly 100 cannot be advanced up the guidewire to the surgical site, the procedure can be aborted before the package for the delivery catheter is opened and the expense of the delivery catheter (and stent) incurred.

Furthermore, if, after the facilitating catheter assembly 100 has been advanced to the surgical site, after the facilitating sheath 105 has been deployed, and after one or more devices passed up the facilitating sheath, it should be discovered that the blood vessel has been damaged (e.g., ruptured, roughened, etc.), the facilitating sheath 105 may be sacrificed, in situ, for conversion into a permanent liner (i.e. an endoluminal graft) for the blood vessel. Thus, serious bleeding is avoided, as well as the need for subsequent repair surgery.

In this case where the facilitating sheath is to be converted into a permanent liner/endoluminal graft, fixation of the permanent liner/graft can be done by standard stenting at its more distal extent. In the case where an aneurysm stent is to be deployed, the aneurysm stent itself can be used to secure the distal end of the permanent liner/graft. In the case where the aneurysm stent is not going to be deployed, standard stenting can be used to secure the distal end of the permanent liner/graft, e.g., in the iliac branch. Proximally, the fixation technique will depend on the length of graft. If the proximal fixation point of the permanent liner/graft is at the opening of the femoral artery in the groin, the permanent liner/graft can be cut to size and sutured in place with standard sutures. Fixation of any shorter length of permanent liner/graft (e.g., within the iliac artery) may be done by standard endoluminal stenting, including a proximal end of the aneurysm stent.

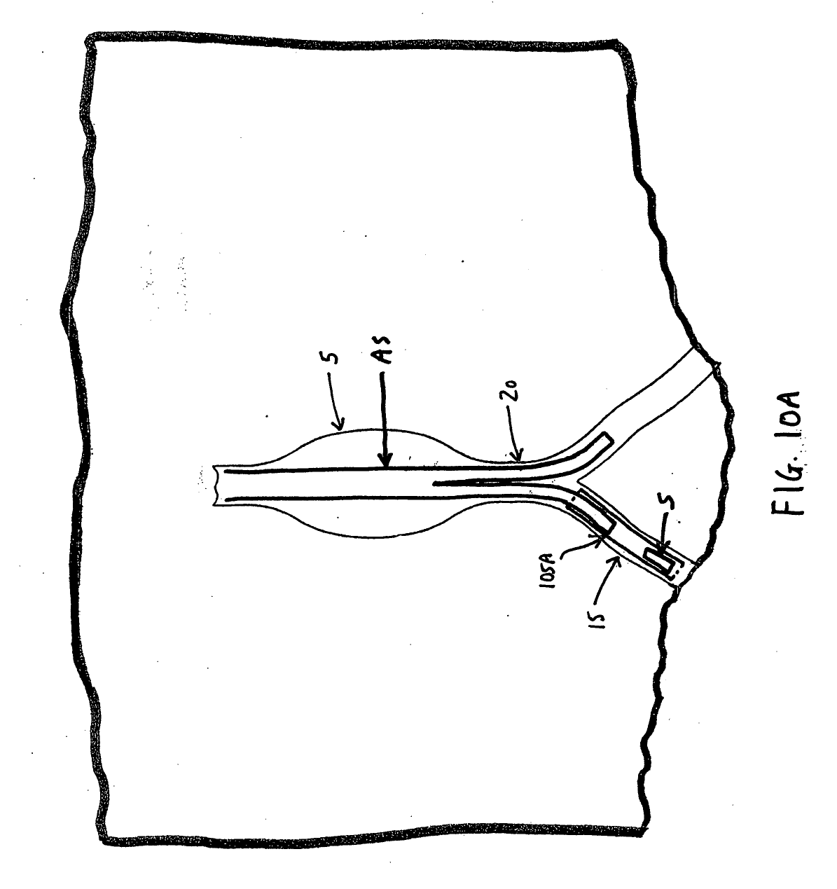

To cut the permanent liner/graft to size within the artery will generally require the use of an endoluminal cutting device. One such device is shown in FIG. 9. More particularly, in FIG. 4 there is shown an endoluminal cutter 130 comprising a pair of concentric cutter tubes, one tube 135 external to the permanent liner/graft 105 and the other tube 140 internal to the permanent liner/graft. The outer tube 135 has an internal shoulder 145 and the inner tube 140 has a front cutting edge 150. Bringing the two tubes together, and then rotating the two tubes in opposite directions, will cut the permanent liner/graft within the lumen of the artery precisely at the required length. Once cut, the proximal end of the graft may be stented for fixation. In this respect it will be appreciated that the permanent liner/graft will be held open after cutting by blood flow, thereby making it easy to stent the permanent liner/graft into position. Alternatively, the permanent liner/graft may be cut after stenting, e.g., by cutting it off proximal to the stented region.

Alternative methods of fixation, e.g., suturing, stapling, etc., and alternative methods of cutting, such as using electrocautery, may also be used.

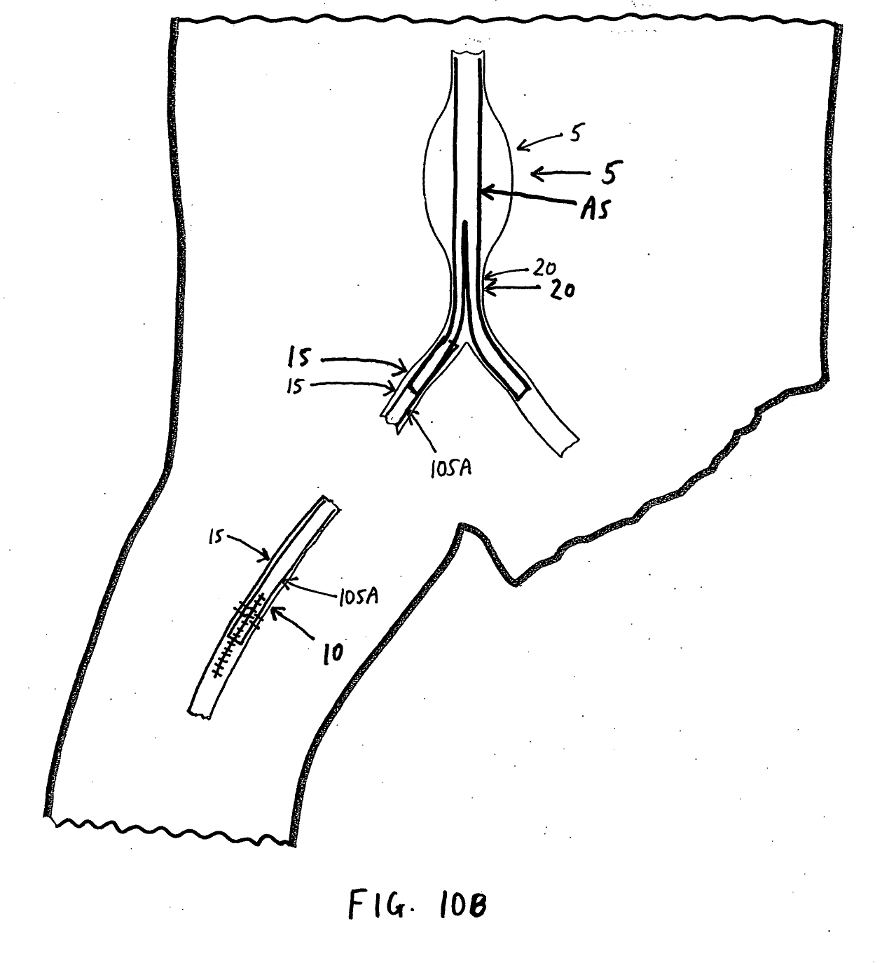

FIG. 10A shows the permanent liner/graft 105A positioned in the vascular system of the patient, with the distal end of permanent liner/graft 105A held in place within the iliac branch by an aneurysm stent AS and the proximal end of the permanent liner/graft 105A held in place within the iliac branch by another stent S.

FIG. 10B shows the permanent liner/graft 105A positioned in the vascular system of the patient, with the distal end of the permanent liner/graft 105A held in place in the iliac branch by an aneurysm stent AS and the proximal end of the permanent liner/graft held in place within the femoral artery by sutures.

In the foregoing description, the present invention has been discussed in the context of a procedure to repair on aortic aneurysm. However, it should also be appreciated that the invention may be used in connection with any procedure being performed through a vessel, e.g., a procedure being conducted through or on the coronary, renal, visceral, cerebral and/or other vessels.

1. A method for advancing a working instrument through the vascular system of a patient, said method comprising the steps of:
advancing a facilitating catheter assembly within the vascular system of a patient, wherein said facilitating catheter assembly is formed out of a implantable graft material; and
advancing the working instrument through said facilitating catheter assembly.

2. A method according to claim 1 wherein said interior of said facilitating catheter assembly comprises a material having a relatively low coefficient of friction.

3. A method according to claim 1 wherein said facilitating catheter assembly comprises a facilitating sheath and a companion sheath, said facilitating sheath being disposed within said companion sheath, said companion sheath including means for opening said companion sheath so as to release said facilitating sheath, and said facilitating sheath being formed out of implantable graft material.

4. A method according to claim 3 wherein said facilitating sheath is released from said companion sheath after said facilitating catheter assembly has been advanced within the vascular system of a patient and before the working instrument is advanced through said facilitating sheath.

5. A method according to claim 3 wherein said companion sheath is removed after said facilitating sheath has been released from said companion sheath.

6. A method according to claim 3 wherein said facilitating sheath is configured to have (i) a first diameter when it is disposed within said companion sheath, and (ii) a second, larger diameter when it is released from said companion sheath.

7. A method according to claim 3 wherein at least a portion of said facilitating sheath is secured in position within the vascular system of a patient.

8. A method according to claim 7 wherein said facilitating sheath is cut prior to at least a portion of said facilitating sheath being secured in position within the vascular system of a patient.

9. A method according to claim 7 wherein said at least a portion of said facilitating sheath is secured in position using a stent.

10. A method according to claim 1 wherein said facilitating catheter assembly is advanced within the vascular system of a patient by advancing it over a guidewire.

11. A facilitating catheter assembly for use in for advancing a working instrument through the vascular system of a patient, said facilitating catheter assembly comprising a facilitating sheath and a companion sheath, said facilitating sheath being disposed within said companion sheath, said companion sheath including means for opening said companion sheath so as to release said facilitating sheath, and said facilitating sheath being formed out of implantable graft material.

12. Apparatus according to claim 11 wherein said interior of said facilitating sheath comprises a material having a relatively low coefficient of friction.

13. Apparatus according to claim 11 wherein said facilitating sheath is configured to have (i) a first diameter when it is disposed within said companion sheath, and (ii) a second, larger diameter when it is released from said companion sheath.

14. Apparatus according to claim 11 wherein said facilitating catheter assembly is configured to be advanced within the vascular system of a patient by advancing it over a guidewire.

15. A method for advancing a working instrument through the vascular system of a patient, said method comprising the steps of:
advancing a facilitating catheter assembly within the vascular system of a patient, wherein said facilitating catheter assembly is constructed so as to (i) have a first diameter which is less than the diameter of the working instrument, and (ii) be capable of expanding to a second diameter which is at least as large as the diameter of the working insturment; and
advancing the working instrument through said facilitating catheter assembly.

16. A method according to claim 15 wherein said interior of said facilitating catheter assembly comprises a material having a relatively low coefficient of friction.

17. A method according to claim 15 wherein said facilitating catheter assembly comprises a facilitating sheath and a companion sheath, said facilitating sheath being disposed within said companion sheath, said companion sheath including means for opening said companion sheath so as to release said facilitating sheath, and said facilitating sheath being formed out of implantable graft material.

18. A method according to claim 17 wherein said facilitating sheath is released from said companion sheath after said facilitating catheter assembly has been advanced within the vascular system of a patient and before the working instrument is advanced through said facilitating sheath.

19. A method according to claim 17 wherein said companion sheath is removed after said facilitating sheath has been released from said companion sheath.

20. A method according to claim 17 wherein said facilitating sheath is configured to have (i) a first diameter when it is disposed within said companion sheath, and (ii) a second, larger diameter when it is released from said companion sheath.

21. A method according to claim 17 wherein at least a portion of said facilitating sheath is secured in position within the vascular system of a patient.

22. A method according to claim 21 wherein said facilitating sheath is cut prior to at least a portion of said facilitating sheath being secured in position within the vascular system of a patient.

23. A method according to claim 21 wherein said at least a portion of said facilitating sheath is secured in position using a stent.

24. A method according to claim 15 wherein said facilitating catheter assembly is advanced within the vascular system of a patient by advancing it over a guidewire.

25. A facilitating catheter assembly for use in advancing a working instrument through the vascular system of a patient, said facilitating catheter assembly being constructed so as to (i) have a first diameter which is less than the diameter of the working instrument, and (ii) be capable of expanding to a second diameter which is at least as large as the diameter of the working instrument.

26. Apparatus according to claim 25 wherein said facilitating catheter assembly comprises a facilitating sheath and a companion sheath, said facilitating sheath being disposed within said companion sheath, said companion sheath including means for opening said companion sheath so as to release said facilitating sheath, and said facilitating sheath being constructed so as to (i) have a first diameter which is less than the diameter of the working instrument, and (ii) be capable of expanding to a second diameter which is at least as large as the diameter of the working instrument.

27. Apparatus according to claim 26 wherein said interior of said facilitating sheath comprises a material having a relatively low coefficient of friction.

28. Apparatus according to claim 26 wherein said facilitating sheath is configured to have (i) a first diameter when it is disposed within said companion sheath, and (ii) a second, larger diameter when it is released from said companion sheath.

29. Apparatus according to claim 26 wherein said facilitating catheter assembly is configured to be advanced within the vascular system of a patient by advancing it over a guidewire.
